(19) United States
(12) Patent Application Publication
(10) Pub. No.: US 2023/0190589 A1
(43) Pub. Date: Jun. 22, 2023

(54) COSMETIC GEL COMPOSITION COMPRISING PARTICLES

(71) Applicant: L'OREAL, Paris (FR)

(72) Inventors: Hungta LIN, Teaneck, NJ (US); Siliu TAN, Westfield, NJ (US); Leandro VALERIANO de CARVALHO, Rio de Janeiro (BR)

(73) Assignee: L'OREAL, Paris (FR)

(21) Appl. No.: 17/556,022

(22) Filed: Dec. 20, 2021

(51) Int. Cl. A61K 8/04; A61K 8/81; A61Q 19/00

(52) U.S. Cl. CPC A61K 8/042; A61K 8/8147; A61Q 19/00; A61K 2800/87; A61K 2800/412

(57) ABSTRACT

The present invention relates to a gel composition that includes water; at least one C2-C5 alcohol; at least one copolymer formed from sulfonic acid acrylic monomer; at least one acrylic thickening polymer free of sulfonated monomers; and a particulate portion comprising a majority fraction of substantially spherical particulates.

### FIELD OF THE INVENTION

The present invention relates to a gel composition as well as cosmetic systems comprising the gel composition and methods for making-up and enhancing the appearance of keratinous materials using the gel composition.

### DISCUSSION OF THE BACKGROUND

Although cosmetic compositions such as gel compositions are known, there remains a need for improved ways to make-up and enhance the appearance of keratinous materials using such compositions.

The present inventors have found that prior gel compositions for use with porous applicators can suffer from the drawback of demonstrating a poor ability to express the entire composition through the pores of the applicator (i.e., “restitution”) and/or have poor phase stability. The present invention overcomes one or more of these drawbacks.

### SUMMARY OF THE INVENTION

The present invention relates to a cooling gel composition that includes water; at least one C2-C5 alcohol; at least one copolymer formed from sulfonic acid acrylic monomer; at least one acrylic thickening polymer free of sulfonated monomers; and a particulate portion comprising a majority fraction of substantially spherical particulates having an average particle size less than about 20 microns.

According to another aspect the present invention relates to a cosmetic system that includes an applicator that includes a container and at least one porous material in the container, wherein the porous material has different pore sizes in different portions of the Material. The system further includes a gel composition such as the gel composition described above.

According to another aspect the present invention relates to a method for enhancing the appearance of skin that includes applying a gel composition such as the gel composition described above to the skin using an applicator comprising a container and at least one porous material in the container, wherein the porous material has different pore sizes in different portions of the material.

It is to be understood that both the foregoing general description and the following detailed description are exemplary and explanatory only, and are not restrictive of the invention.

### BRIEF DESCRIPTION OF THE DRAWINGS

FIG. 1 depicts an example of a container suitable for use according to embodiments of the present invention.

FIG. 2 is a cross-section of an applicator suitable for use according to embodiments of the present invention, the applicator comprising a base portion of the container, a cap of the container, a storage reservoir, and a porous material.

### DETAILED DESCRIPTION OF THE INVENTION

According to the invention, the term “devoid of” a particular material refers to a composition not containing any of the particular material. So, for example, a composition “devoid of” surfactant does not contain any surfactant. Similarly, a composition “free of” surfactant contains 0.25% by weight of the composition or less of surfactant. A composition “substantially free of” surfactant contains 1% by weight of the composition or less of surfactant.

As used herein, the expression “at least one” means at least one and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

“Film former” or “film forming agent” or “film forming polymer” or “film forming resin” as used herein mean a polymer or resin that leaves a film on the substrate to which it is applied, for example, after a solvent accompanying the film former has evaporated, absorbed into and/or dissipated on the substrate.

“Substituted” as used herein, means comprising at least one substituent. Non-limiting examples of substituents include atoms, such as oxygen atoms and nitrogen atoms, as well as functional groups, such as amine groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphategroups, siloxane groups, and polysiloxane groups. The substituent(s) may be further substituted.

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

As used herein a range of ratios is meant to include every specific ratio within, and combination of subranges between the given ranges.

“Keratinous materials” includes materials containing keratin such as hair, skin, eyebrows, lips and nails, preferably skin.

“Volatile”, as used herein, means having a flash point of less than about 100° C.

“Non-volatile”, as used herein, means having a flash point of greater than about 100° C.

“Gel Crossover Point” (Sol/Gel Point), means the point at which the G″ (loss modulus) intersects the G′ (storage modulus), reported in % strain. It is the point at which a composition goes from a more solid state to a more liquid state. An example of a method for determining gel crossover point is as follows: a Discovery HR-2 Rheometer by TA Instruments can be used, having 40 mm parallel plate geometry on a stainless steel flat peltier plate. The test can be run @ 20° C., with test parameter of angular frequency of 1.0 rad/s and logarithmic sweep: Strain % 0.01 to 1000.0%. 5 points per decade. Results reported in % strain.

“Viscosity,” as used herein, may be measured at a temperature of 25° C., for example, using a Brooksfield viscometer, Model RVT (Brookfield Engineering Laboratories, Inc.) at about 6 revolutions per minute (RPM), at ambient room temperature of about 20 to 25° C.; spindle sizes may be selected in accordance with the standard operating recommendations form the manufacturer, such as a T-Bar No. 34 spindle. Such viscosity readings for gel compositions of the present invention may be less than about 1000 centipoise, such as from about 100 cps to about 800 cps, such as from about 400 cps to about 800 cps when measured accordingly.

The compositions, systems and methods of the present invention can comprise, consist of, or consist essentially of the essential elements and limitations of the invention described herein, as well as any additional or optional ingredients, components, or limitations described herein or otherwise useful.

Gel Composition

According to the present invention, cooling gel compositions that include water; at least one C2-C5 alcohol; at least one copolymer formed from sulfonic acid acrylic monomer; at least one acrylic thickening polymer free of sulfonated monomers; and a particulate portion comprising a majority fraction of substantially spherical particulates substantially spherical particulates having an average particle size less than about 20 microns are provided. The gel composition may be free of surfactants. According to certain embodiments, the gel compositions have a gel crossover point ranging from about 35% to about 85% strain, preferably from about 40% to about 80% strain, and preferably from about 45% to about 75% strain, including all ranges and subranges therebetween.

For purposes of the present invention, the “color cosmetic composition” referred to herein can be any suitable cosmetic composition containing at least one coloring agent which can provide color to a keratinous material to which it has been applied.

C2-C5 Alcohol and Water (Hydro-Alcoholic Solvent Component)

According to the present invention, the gel composition includes at least one C2-C5 alcohol. Suitable C2-C5 alcohols include ethanol, propanol, butanol, pentanol, isopropanol, isobutanol and isopentanol. Ethanol is particularly preferred.

Preferably, the C2-C5 alcohol(s) is/are present in the gel compositions of the present invention in amounts ranging from about 0.5% to about 30%, preferably from about 4% to about 30%, preferably from about 5% to about 30%, preferably from about 10% to about 30%, preferably from about 20% to about 30%, by weight, based on the total weight of the composition, including all ranges and subranges in between.

According to the present invention, the gel composition include water. Preferably, the gel compositions comprise from about 40% to about 90% water, preferably from about 50% to about 85% water, and preferably from about 60% to about 80% water, by weight, based on the total weight of the composition, including all ranges and subranges therebetween.

Preferably, the solvent component of the gel compositions of the present invention consists essentially of, or consists of, water and C2-C5 alcohols. That is, the gel composition preferably will not contain any solvent in an amount which has a material adverse effect on the transfer-resistance properties of a color cosmetic composition to which it has been applied.

According to preferred embodiments, the gel compositions of the present invention are devoid of, free of, or substantially free of surfactants. Such surfactants include those amphiphilic molecules capable of depressing surface tension of water to less than about 50 mN/m, such as less than about 40 mN/m, when present in a concentration by weight in deionized water of 0.25% at 25° C., particularly those having a molecular weight of less than about 600 daltons, and more particularly foaming surfactants.

According to preferred embodiments, the gel compositions of the present invention are devoid of, free of, or substantially free of silicones.

According to preferred embodiments, the gel compositions of the present invention are devoid of, free of, or substantially free of film formers.

According to preferred embodiments, the gel compositions of the present invention are devoid of, free of, or substantially free of coloring agents.

According to preferred embodiments, the gel compositions of the present invention are devoid of, free of, or substantially free of at least one of coloring agents, surfactants, silicones and film formers.

Particulate Portion

According to the present invention, the gel composition includes a particulate portion. The particulate portion consists of any of various finely divided solid materials that are suspended or otherwise stabilized in the gel composition.

The inventors have found that, in order to enhance the expression of the particles through the porous applicator, it is important that the particulates chosen in the composition include substantially spherical particles having an average particle size less than about 20 microns.

By “substantially spherical,” it is meant an essentially spherical shape, for example in the form of beads (hollow or solid), Suitable examples of substantially spherical particles include certain silica aerogels. Silica aerogels are porous materials obtained by replacing (by drying) the liquid component of a silica gel with air. They are generally synthesized via a sol-gel process in liquid medium and then dried, usually by extraction of a supercritical fluid, the one most commonly used being supercritical CO₂. This type of drying makes it possible to avoid shrinkage of the pores and of the material. The sol-gel process and the various drying processes are described in detail in Brinker C J., and Scherer G. W., Sol-Gel Science: New York: Academic Press, 1990. Silica aerogels, in general, have been disclosed in U.S. Pat. No. 9,320,689, the entire contents of which is hereby incorporated by reference.

Preferably, the aerogels used according to the present invention are hydrophobic silica aerogels. The term “hydrophobic silica” means any silica whose surface is treated with silylating agents, for example halogenated silanes such as alkylchlorosilanes, siloxanes, in particular dimethylsiloxanes such as hexamethyldisiloxane, or silazanes, so as to functionalized the OH groups with silyl groups Si—Rn, for example trimethylsilyl groups. Hydrophobic silica aerogels particles surface-modified with trimethylsilyl groups (trimethylsilyl silica) are preferred. As regards the preparation of hydrophobic silica aerogels particles that have been surface-modified by silylation, (“silica silylate”), reference may be made to document U.S. Pat. No. 7,470,725, the entire contents of which is hereby incorporated by reference.

Examples of substantially spherical silica aerogels having an average particle size less than about 20 microns include AIRLICA TL-10 from Tokuyama Corporation or CAB-O-SIL TS-530 from Cabot Corporation.

While substantially spherical silica aerogel particulates having an average particle size less than about 20 microns are exemplified above, the substantially spherical particulates having an average particle size less than about 20 microns need not be silica aerogel. They may, for example be other spherical silicas such as Silica Balloon BA4 available from JGC Catalysts and Chemicals. This silica has an oil absorption 40 mL/100-gram powder and an average particle size of about 4 μm. Other microspheres (beads) made from glass or polymers are also suitable. In one notable embodiment, the spherical particles having an average particle size less than about 20 microns comprise silica. In other notable embodiments, the spherical particles are silica aerogel particles.

While the particulate portion may include particulates that are not substantially spherical particulates having an average (e.g., median) particle size less than about 20 microns, at least 50% by weight of the particulate material in the particulate portion meets this requirement. Accordingly, the particulate portion “comprises a majority fraction of substantially spherical particulates having an average particle size less than about 20 microns.”

According to certain embodiments, the particulate portion is substantially free of substantially non-spherical particulates. However, if included, such substantially non-spherical particulates may include, for example, perlite; poly(vinyl chloride) powder; pumice; ground fruit kernel shells, such as ground materials derived from apricot kernels or walnut shells; sawdust, wood flour; cellulose, and the like. Other particles which may be included are certain polyethylene beads, and certain nylon particles, such as the Nylon-12.

Preferably, the substantially spherical particulates having an average particle size less than about 20 microns is/are present in the gel compositions of the present invention in amounts ranging from about 0.05% to about 10% by weight, preferably from 0.1% to 2% by weight, and preferably from 0.1% to 1% by weight, and more preferably from 0.25% to 1%, and more preferably from 0.2% to 0.8% by weight including all ranges and subranges therebetween, all weights based on the weight of the composition as a whole. The total percentage of particulates in the gel composition may range from about 0.05% to about 10% by weight, such as from about 0.1% to about 5%, such as from about 0.1% to about 1% by weight.

Copolymer Formed from Sulfonic Acid Acrylic Monomer

In accordance with the present invention, compositions that include at least one copolymer formed from sulfonic acid acrylic monomer are provided.

Preferably, the copolymer formed from sulfonic acid acrylic monomer comprises at least one ethylenically unsaturated monomer containing a sulphonic group, in freeform or partially or totally neutralized form and comprising at least one hydrophobic portion. The hydrophobic portion present in these polymers preferably contains from 6 to 50 carbon atoms, preferably from 6 to 22 carbon atoms, preferably from 6 to 18 carbon atoms and preferably from 12 to 18 carbon atoms, including all ranges and subranges therebetween.

The copolymer formed from sulfonic acid acrylic monomer may be based on at least one ethylenically unsaturated hydrophilic monomer A and on at least one hydrophobic monomer B. Preferably, the monomer A comprises a strong acid function, in particular a sulphonic acid or phosphonic acid function. The hydrophobic monomer B comprises at least one hydrophobic radical, chosen from: saturated or unsaturated C₆-C₁₈ linear alkyl radicals (for example, n-hexyl, n-octyl, n-decyl, n-hexadecyl, n-dodecyl or oleyl); branched alkyl radicals (for example, isostearic) or cyclic alkyl radicals (for example, cyclododecane or adamantane); C₆-C₁₈ fluoro or alkylfluoro radicals (for example, the group of formula —(CH₂)₂—(CF₂)₉—CF₃); a cholesteryl radical or radicals derived from cholesterol (for example, cholesteryl hexanoate); aromatic polycyclic groups, for instance naphthalene or pyrene; and silicone or alkylsilicone or alkylfluorosilicone radicals. Among these radicals, linear and branched alkyl radicals are preferred.

The copolymer formed from sulfonic acid acrylic monomer are preferably water-soluble or water-dispersible in neutralized form. Their viscosity at a concentration of 1% in water, at a shear rate of 1 s⁻¹, at a pH of between 5 and 8, at 25° C., is preferably less than 5 000 mPa·s. Copolymers formed from sulfonic acid acrylic monomer suitable for use in the present invention are described in US 2001/049419 and U.S. Pat. No. 6,645,476, the entire contents of both of which are hereby incorporated by reference.

The copolymer formed from sulfonic acid acrylic monomer may be crosslinked. The crosslinking agents may be chosen from, for example, the polyolefinically unsaturated compounds commonly used for crosslinking polymers obtained by free-radical polymerization. According to one preferred embodiment of the invention, the crosslinking agent is chosen from methylenebisacrylamide, allyl methacrylate or trimethylolpropane triacrylate (TMPTA). The degree of crosslinking preferably ranges from 0.01 mol % to 10 mol %, and preferably from 0.2 mol % to 2 mol %, relative to the polymer, including all ranges and subranges therebetween. Preferred amphiphilic polymers are chosen from crosslinked or non-crosslinked amphiphilic polymers of 2acrylamido-2-methylpropanesulphonic (AMPS) acid and of at least one ethylenically unsaturated monomer comprising at least one hydrophobic portion containing from 6 to 30 carbon atoms, preferably from 6 to 22 carbon atoms, preferably from 6 to 18 carbon atoms and preferably from 12 to 18 carbon atoms, including all ranges and subranges therebetween.

Suitable copolymers formed from sulfonic acid acrylic monomers include crosslinked acrylamide/sodium acrylamido-2-methylpropanesulfonate (AMPS) copolymers, such as that used in the commercial product Sepigel 305 (CTFA name: Polyacrylamide/C₁₃-C₁₄ Isoparaffin/Laureth-7) or that used in the commercial product sold under the name Simulgel 600 (CTFA name: Acrylamide/Sodium Acryloyldimethyltaurate/Isohexadecane/Poly sorbate-80) by the company SEPPIC; copolymers of AMPS® and of vinylpyrrolidone or vinylformamide, such as that used in the commercial product sold under the name Aristoflex AVC® by the company Clariant (CTFA name: Ammonium acryloyldimethyltaurate/VP copolymer) but neutralized with sodium hydroxide or potassium hydroxide; copolymers of AMPS and of sodium acrylate, for instance the AMPS/sodium acrylate copolymer, such as that used in the commercial product sold under the name Simulgel EG® by the company SEPPIC or under the trade name Sepinov EM (CTFA name: Hydroxyethyl acrylate/sodium acryloyldimethyl taurate copolymer); copolymers of AMPS and of hydroxyethyl acrylate, for instance the AMPS/hydroxyethyl acrylate copolymer, such as that used in the commercial product sold under the name Simulgel NS® by the company SEPPIC (CTFA name: Hydroxyethyl acrylate/sodium acryloyldimethyltaurate copolymer (and) squalane (and) polysorbate 60), or such as the product sold under the name Sodium acrylamido-2-methylpropanesulfonate/hydroxyethyl acrylate copolymer, such as the commercial product Sepinov EMT 10 (INCI name: Hydroxyethyl acrylate/sodium acryloyldimethyl taurate copolymer); Ammonium Acryloyldimethyltaurate/Beheneth-25 Methacrylate Crosspolymer (Aristoflex HMB from Clariant) (crosslinked ethoxylated AMPS/behenyl methacrylate); Ammonium Acryloyldimethyltaurate/Steareth-25 Methacrylate Crosspolymer (Aristoflex HMS) (ethoxylated copolymer of AMPS/stearyl methacrylate crosslinked with trimethylol triacrylate), Aristoflex SNC (crosslinked ethoxylated AMPS/C16-C18), Aristoflex LNC (noncrosslinked AMPS/C12-C14), and mixtures thereof.

The at least one copolymers formed from sulfonaic acid acrylic monomers may be present in a concentration by weight ranging from about 0.1% to about 20%, such as from about any of 0.1%, 0.25, 0.5%, or 1% by weight to about any of 1%, 1.5%, or 2% by weight with respect to the total weight of the composition.

Acrylic thickening polymer free of sulfonated monomers

In accordance with the present invention, compositions that include at least one acrylic thickening polymer free of sulfonated monomers are provided. The acrylic thickening polymer free of sulfonated monomers is formed from at least one ethylenically unsaturated monomers, each of which is free of sulfonated moieties. According to certain notable embodiments, the acrylic thickening polymer free of sulfonated monomers is at least partially crosslinked.

Preferably, the acrylic thickening polymer free of sulfonated monomers is an acrylic emulsion polymer (formed from emulsion polymerization). According to certain embodiments, the acrylic thickening polymer free of sulfonated monomers is an alkali-swellable acrylic emulsion polymer. The thickening effect from this polymer may be from neutralization of the protonated carboxyl groups which then ionize, thereby expanding the polymer due to charge repulsion. In certain embodiments, the acrylic thickening polymer free of sulfonated monomers is further hydrophobically modified, such as with one or mor alkyl groups. In certain other embodiments, the acrylic thickening polymer free of sulfonated monomers is free of hydrophobic modification.

Suitable acrylic thickening polymer free of sulfonated monomers include CARBOPOL polymers such as CARBOPOL 2984, CARBOPOL 5984, CARBOPOL 940, CARBOPOL 941, CARBOPOL 980, CARBOPOL 981, CARBOPOL Clear Polymer, CARBOPOL SC-200, CARBOPOL SC-800, CARBOPOL Silk-100, CARBOPOL Ultrez-10, CARBOPOL Aqua SF-1. CARBOPOL polymer are available from Lubrizol.

According to preferred embodiments the acrylic thickening polymer free of sulfonated monomers is CARBOPOL Aqua SF-1, a lightly-crosslinked alkali emulsion polymer.

The at least one acrylic thickening polymer free of sulfonated monomers may be present in a concentration by weight ranging from about 0.1% to about 20%, such as from about any of 0.1%, 0.25% 0.5%, or 1% by weight to about any of 1%, 1.5%, or 2% by weight with respect to the total weight of the composition.

Additional Ingredients

According to preferred embodiments, the gel compositions can comprise additional ingredients typically included in cosmetic compositions. A non-exhaustive discussion of such ingredients is set forth below.

According to preferred embodiments, the gel compositions may optionally further comprise as least one film-forming agent (that do not meet the requirements for either the at least one acrylic thickening polymer free of sulfonated monomers or the at least one copolymers formed from sulfonaic acid acrylic monomers). Examples include silicone film-forming agent selected from the group consisting of silicone resins, polyorganosiloxane copolymers, and mixtures thereof. Also preferably, the silicone resin is selected from the group consisting of siloxysilicate resins, silsesquioxane resins, and mixtures thereof. In certain embodiments, the gel compositions of the present invention are devoid of or free of the film-forming agents described in this paragraph.

Other additional ingredients optionally included in the gel composition include glycols such as glycerin, butylene-glycol, hexylene glycol or propylene glycol; fragrance; a preserving agent; neutralizing agents such as alkalis including sodium hydroxide; cooling agents such as methyl diisopropyl propionamide; cosmetically active agents and/or dermatological active agents such as, for example, emollients, moisturizers, vitamins, hydroxy acids such salicylic acid, and medicaments.

According to preferred embodiments, methods for enhancing the appearance of a skin include optionally applying a color cosmetic composition to the keratinous material; and (2) applying the gels compositions of the present invention either (a) the color cosmetic composition or (b) directly to the skin, are provided.

According to certain embodiments, the gel composition of the present invention includes less than about 5% solids (percent solids is determined starting at 100% and subtracting all volatile solvents and water. One skilled in the art will recognize this can be also measured placing a small amount of material in a suitable flat container and allowing it to sit at 105° C. until a steady mass is obtained. leaving behind the condensed phase material that.

According to preferred embodiments, gel compositions of the present invention comprise from about 50% by weight to about 85% by weight of water; from about 5% by weight to about 30% by weight of water at least one C2-C5 alcohol; from about 0.1% to about 2% by weight of at least one copolymer formed from sulfonic acid acrylic monomer; from about 0.1% to about 2% by weight at least one acrylic thickening polymer free of sulfonated monomers; and from about 0.1% to 2% by weight a particulate portion comprising a majority fraction of substantially spherical particulates. This gel composition may include less than 5% solids and may be substantially free of surfactants, such as those amphiphilic molecules capable of depressing surface tension of water to less than about 50 mN/m, such as less than about 40 mN/m, when present in a concentration by weight in deionized water of 0.25% at 25° C., particularly those having a such materials having molecular weight of less than about 600 daltons. This gel composition may have a viscosity of less than about 1000 centipoise, such as 100 cps to 800 cps, when measured at 6 rpm using a T-Bar No. 34 spindle. This gel composition may be substantially free of substantially nonspherical particulates.

Applicator

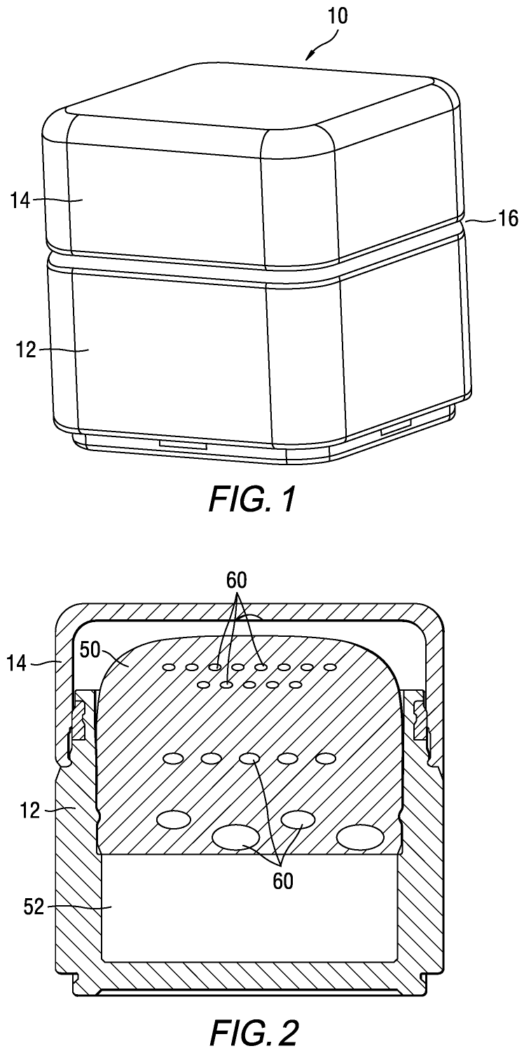

According to the present invention, cosmetic systems of the present invention comprise at least one applicator. According to preferred embodiments, as depicted in FIG. 1, the applicator comprises a container (10) and at least one porous material (50) in the container (10).

According to preferred embodiments, the container (10) can be made of any substance suitable for housing the porous material such as, for example, plastic, glass, metal, wood, etc. Further, the container substance can be hard (does not deform upon application of pressure), resilient (slightly deforms upon application of pressure but returns to its original form after application pressure stops) or soft (deforms upon application of pressure). Cross-sections of the container can be of shape such as, for example, square, rectangular, circular, elliptical, oval, etc.

According to preferred embodiments, the container is of a size and shape which can be held comfortably and/or ergonomically in a hand during application. For example, preferred containers comprise a cross-section having a width of 1.5 cm to 4.5 cm and a length of 1.5 cm to 4.5 cm, preferably a width of 2 cm to 4 cm and a length of 2 cm to 4 cm, and preferably a width of 2.5 cm to 3.5 cm and a length of 2.5 cm to 3.5 cm, including all ranges and subranges therebetween. According to preferred embodiments, the length and width of the container (cross-section) are in the shape of a square, and have a length and width of about 2.5 cm to about 3 cm. Such preferred embodiments as described in this paragraph can provide a combination of quick application of product and ability to reach sensitive areas like skin under the eyes and around the nose.

According to preferred embodiments, the container comprises a base portion (12) and a cap (14). During use of the cosmetic systems of the present invention, the cap (14) is removed from the base portion (12) to expose the porous material (50) in the base portion (12), and the porous material (50) is contacted with keratinous material as described more fully below. The cap (14) can be detachable from the base portion (12), or the cap (14) can be connected to the base portion (12) through a hinge or other known attachment structure (16).

According to preferred embodiments, the porous material (50) can either be removable (for example, in a reusable system) or not removable (for example, in a single use system) from the base portion (12) of the container (10).

According to preferred embodiments, the porous material (50) has different sizes of pores (60) in different portions of the material. Preferably, the porous material (50) has a pore (60) size gradient in which pore size increases from one side or portion of the material to the opposite side or portion of the material. Preferably, as depicted in FIG. 2, the porous material has a pore size gradient which goes from large pore size at the side or portion of the porous material at the bottom of the base portion of the container (that is, the side or portion of the porous material facing the bottom of the base portion of the container) to small pore size at the side or portion of the porous material to be contacted with keratinous material (that is, the side or portion of the porous material facing outward from the base portion of the container). Such a gradient can be made, for example, by serially combining layers of different, desired porosities, or by sintering or molding materials at specific rates, temperatures and pressure as is known in the art.

According to preferred embodiments, the porous material can be made through cast-molding processes as is known in the art. Preferably, if the porous material is made through cast-molding processes, the surface of the mold used to cast and mold the porous material has a texture depth of 0.03 inches or less, including all ranges and subranges therebetween such as, for example, less than 0.01 inches, less than 0.0005 inches, from about 0.01 inches to about 0.03 inches, and from about 0.0001 inches to about 0.01 inches. Preferably, the texture corresponds to that of MT11000 according to the Moldtech book standard. Preferred methods of making the porous material include the processes set forth in U.S. Pat. No. 6,030,558, the entire contents of which is hereby incorporated by reference.

Although not wanting to be bound by any particular theory, it is currently believed that the preferred pore size differences described above allow for the gel composition to move from the base portion of the container to the portion of the porous material to be contacted with keratinous material through wicking and/or capillary action.

According to preferred embodiments, the portion of the porous material to be contacted with keratinous material has an average pore size of about 30 to 80 microns, preferably about 35 to 75 microns, preferably about 40 to 70 microns, and preferably about 45 to 65 microns, including all ranges and subranges therebetween. Preferably, the portion of the porous material to be contacted with keratinous material constitutes no more than half (50%) of the porous material, preferably no more than a third (33%) of the porous material, and preferably no more than a quarter (25%) of the porous material.

According to preferred embodiments, the portion of the porous material at the bottom of the base portion of the container has an average pore size of about 90 to 200 microns, preferably about 95 to 190 microns, preferably about 100 to 170 microns, and preferably about 110 to 160 microns, including all ranges and subranges therebetween. Preferably, the portion of the porous material at the bottom of the base portion of the container constitutes no more than bottom half (50%) of the porous material, preferably no more than a third (33%) of the porous material, and preferably no more than a quarter (25%) of the porous material.

It is to be understood that the porous material may optionally further contain at least one mid-portions between the portion at the bottom of the base portion of the container and the portion to be contacted with keratinous material. Preferably, the at least one mid-portions contain average pore size(s) between those in the portion of the porous material at the bottom of the base portion of the container and those in the portion of the porous material to be contacted with keratinous material. However, it should be understood that it is not necessary that the average pore size(s) in the mid-portion(s), if present, are between the pore sizes of the other portions of the porous material.

According to preferred embodiments, the porous material is soft and/or not abrasive. Preferably, the porous material is a porous polymeric material, or a sponge-like material. Preferably, the porous material is made out of at least one thermoplastic polymers. Suitable examples of such thermoplastic polymers include, but are not limited to, polyethylene (PE), polypropylene (PIP), polyethylene terephthalate (PET), polytetrafluoroethylene (PTFE), ethyl vinyl acetate (EVA), polyvinylidene fluoride (PVDF), polycarbonate (PC), acrylinitrile-butadiene-styrene (ABS), and mixtures thereof. Particularly preferred thermoplastic polymers include low density polyethylene (for example, polyethylene having a density range of 0.910-0,940 g/cm³) and polyethylene terephthalate.

Also preferably, to improve soft and/or non-abrasive properties of the porous material, the porous material has rounded edges. According to preferred embodiments, the porous material can also act as a reservoir for the gel composition. Preferably, the porous material contains enough gel composition to deliver product for at 30 days, preferably at least 60 days, and preferably at least 90 days.

According to preferred embodiments of the present invention, the applicator further contains a storage reservoir (52) for the gel composition. If present, the storage reservoir (52) is preferably located between the base portion of the container (12) and the porous material (50) and is in contact with the porous material (50) such that the storage reservoir supplies gel composition to the porous material (50).

Preferably, the storage reservoir is a porous, low-density layer of compressed fibers which stores gel composition. Preferably, the fibers are made out of at least one thermoplastic polymers. Suitable examples of such thermoplastic polymers include, but are not limited to, polyethylene (PE), polypropylene (PP), polyethylene terephthalate (PET), polytetrafluoroethylene (PTFE), ethyl vinyl acetate (EVA), polyvinylidene fluoride (PVDF), poly carbonate (PC), acrylonitrile-butadiene-styrene (ABS), and mixtures thereof. Particularly preferred thermoplastic polymers include low density polyethylene (for example, polyethylene having a density range of 0.910-0.940 g/cm³) and polyethylene terephthalate.

According to preferred embodiments, the compressed fibers have a density from about 0.04 g/cc to about 0.08 g/cc, preferably from about 0.05 g/cc to about 0.07 g/cc, and preferably about 0.06 g/cc. Also preferably, the compressed fibers are capable of holding about 67% to about 85% of its volume in gel composition, preferably about 70% to about 83%, and preferably from about 77% to about 82%, including all ranges and subranges therebetween.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

The present invention will be better understood from the examples which follow. The examples are intended to be nonrestrictive and explanatory only, with the scope of the invention defined by the claims.

### Example 1—Invention Compositions

The following composition can be prepared, as shown in Table 1:

TABLE 1

| | Concentration by |
|---|---|
| INCI US | Weight (%) |
| AMMONIUM | 0.25-1.5 |
| POLYACRYLOYLDIMETHYLTAURATE | |
| ACRYLATES COPOLYMER | 0.25-3.0 |
| SILICA SILYLATE (substantially spherical) | 0.25-1.0 |
| ALCOHOL DENAT. (ethanol) | 4-30 |
| WATER | 60-80 |
| ADDITIONAL INGREDIENTS* | 1-3 |

*Including only active compounds including salicylic acid; glycols; methyl diisopropyl propionamide; pH adjuster; and fragrance.

### Example 2—Restitution and Stability Testing

The following compositions, as shown in Table 2 were prepared by mixing the ingredients listed below as well as water, ethanol, and additional ingredients and allowing to equilibrate. Sodium hydroxide was used as a pH adjuster to adjust the pH to between 4.5 and 7.0. The balance of ingredients (q.s.) was done by adjusting water concentration. The concentrations of water, ethanol, and additional ingredients were consistent with the ranges provided in Table 2, below:

TABLE 2

| Ingredient | Ex. 1 | Ex. 2 | Comp. 1 | Comp. 2 |
|---|---|---|---|---|
| Water | q.s. | q.s. | q.s. | q.s. |
| Ethanol | 30% | 20% | 30% | 30% |
| Ammonium | 0.6% | 0.6% | — | 0.80% |
| Polyacryloyldimethyltaurate | | | | |
| Acrylates Copolymer | 0.75% | 0.75% | — | — |
| Carbomer | | | 1.07% | |
| Silica Silylate (spherical) | 0.5% | 0.5% | 0.25% | 0.5% |
| Additional Ingredients | 1.7% | 1.7% | 1.7% | 1.6% |
| Restitution (%) | 86 | 85 | 48 | 43 |
| Viscosity (6 rpm, T-Bar No. | 470 | 550 | 6240 | 890 |
| 34) in cps | | | | |
| Stability | Pass | Pass | Fail | Fail |

Restitution testing was performed by loading about 500 milligrams of the composition into a porous applicator consistent with embodiments described herein, such as a porous applicator described in FIG. 2 and having a portion of the porous material to be contacted with keratinous material has an average pore size of about 30 to about 80 microns and a portion of the porous material at the bottom of the base portion of the container having an average pore size of about 90 to 200 microns.

Applications were made by dabbing the applicator 20 times, for 5 sec increments, onto a CURAD non-stick adhesive pad having a 496 g mass provided atop the applicator in order to resemble consumer application force. The % restitution was calculated as (initial mass of composition inside applicator—final mass of composition inside the applicator)/(initial mass of composition inside applicator) and expressed as a percent.

Stability testing was performed by placing composition into a 4 oz. jar and putting the samples into different chambers to test for freeze thaw (e.g., 10 cycles of 20° C. to 20° C.); or constant temperature exposure: 50° C. (one month); and/or 5° C., 25° C., 50° C. or 37° C. for two months. If the samples showed significant color, pH, viscosity change or phase separation, a failure was recorded.

It can be seen that inventive examples, Ex. 1 and Ex. 2 provide excellent restitution and stability, whereas comparative examples have poor restitution and fail stability.

### Example 3—Restitution of Different Particulates

In order to determine whether different particulates would travel through the pores of a porous applicator, six samples were evaluated using a porous applicator consistent with embodiments of the invention described herein. The six formulas included consistent with those shown in Table 1. Each included different particles as shown in Table 3, below. The results of optical microscopy analysis (using Leica DM2500 & DM2500 LED optical microscopes available from Leica Microsystems) to examine the presence of particulates that were able to travel through the pores of the applicator are also shown.

TABLE 3

| | | Average Size | | Microscope |
|---|---|---|---|---|
| Reference | Particulates | (microns) | Shape | Analysis |
| A | Microcrystalline | 2-60 | rectangular | Not visible |
| | cellulose | | | |
| B | Microcrystalline | <15 | rectangular | Not visible |
| | cellulose | | | |
| C | Perlite | 25 | rectangular/ | Not visible |
| | | | polygonal | |
| D | Perlite | 4 | rectangular/ | Not visible |
| | | | polygonal | |
| E | Silica silylate | 5-15 | spherical | Visible |
| F | Silica silylate | 10 | spherical | Visible |

The results indicate that spherical particulates were able to travel through the applicator, whereas others were not.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1. A gel composition comprising:
water;
at least one C2-C5 alcohol;
at least one copolymer formed from sulfonic acid acrylic monomer;
at least one acrylic thickening polymer free of sulfonated monomers; and
a particulate portion comprising a majority fraction of substantially spherical particulates having an average particle size less than about 20 microns.

2. The gel composition of claim 1, wherein the at least one copolymer formed from sulfonic acid acrylic monomer is selected from the group consisting of Ammonium Acryloyldimethyltaurate/VP Copolymer, Ammonium Acryloyldimethyltaurate/Beheneth-25 Methacrylate Crosspolymer, Ammonium Acryloyldimethyltaurate/Steareth-25 Methacrylate Crosspolymer, crosslinked ethoxylated AMPS/C16-C18, noncrosslinked AMPS/C12-C14, and mixtures thereof.

3. The gel composition of claim 1, wherein the at least one acrylic thickening polymer free of sulfonated monomers is an alkali-swellable crosslinked acrylic emulsion polymer.

4. The gel composition of claim 1, wherein the particulate portion comprising a majority fraction of substantially spherical particulates is substantially free of substantially non-spherical particulates.

5. The gel composition of claim 1, wherein the at least one C2-C5 alcohol is ethanol.

6. The gel composition of claim 1, wherein gel composition has a viscosity of less than about 1000 centipoise when measured at 6 rpm using a T-Bar No. 34 spindle.

7. The gel composition of claim 1, wherein at least one C2-C5 alcohol is present in a concentration by weight from about 5% to about 30%.

8. The gel composition of claim 1, wherein the water is present in a concentration by weight from about 50% to about 85%.

9. The gel composition of claim 1, wherein the at least one copolymer formed from sulfonic acid acrylic monomer and the at least one acrylic thickening polymer free of sulfonated monomers are each present in respective concentrations by weight that are from about 0.1% to about 2%.

10. The gel composition of claim 1, wherein the at least one copolymer formed from sulfonic acid acrylic monomer and the at least one acrylic thickening polymer free of sulfonated monomers are each present in respective concentrations by weight that are from about 0.25% to about 1%.

11. The gel composition of claim 1, wherein the at least one copolymer formed from sulfonic acid acrylic monomer is present in a concentration by weight that is less than the concentration by weight of the at least one acrylic thickening polymer free of sulfonated monomers.

12. The gel composition of claim 1, wherein the gel composition consists of the water, the at least one C2-C5 alcohol, the at least one copolymer formed from sulfonic acid acrylic monomer, the at least one acrylic thickening polymer free of sulfonated monomers, the particulate portion comprising a majority fraction of substantially spherical particulates, and additional ingredients, wherein the additional ingredients are collectively present in a concentration by weight from 0% to about 5%.

13. The gel composition of claim 11, wherein the additional ingredients are collectively present in a concentration by weight from 0% to about 2.5%.

14. The gel composition of claim 11, wherein the additional ingredients include salicylic acid.

15. The gel composition of claim 1, wherein the gel composition has a gel crossover point ranging from about 35% to about 85% strain.

16. A cosmetic system comprising:
an applicator comprising a container and at least one porous material in the container, wherein the porous material has different pore sizes in different portions of the material; and
a gel composition comprising:
water;
at least one C2-C5 alcohol;
at least one copolymer formed from sulfonic acid acrylic monomer;
at least one acrylic thickening polymer free of sulfonated monomers; and
a particulate portion comprising a majority fraction of substantially spherical particulates.

17. The cosmetic system of claim 16, wherein the applicator comprises a portion for contacting the skin having an average pore size of about 30 to 80 microns.

18. A method for enhancing the appearance of skin comprising:
applying the gel composition of claim 1 to the skin using an applicator comprising a container and at least one porous material in the container, wherein the porous material has different pore sizes in different portions of the material.
